(19) United States
(12) Patent Application Publication
(10) Pub. No.: US 2025/0204853 A1
(43) Pub. Date: Jun. 26, 2025

(54) DISPOSABLE SENSOR ARRAY WEARABLE DEVICE SLEEVE SYSTEM AND METHOD

(71) Applicant: BRAGI GmbH, München (DE)

(72) Inventors: Peter Vincent Boesen, München (DE); Jake Berry Turner, München (DE)

(73) Assignee: BRAGI GmbH, München (DE)

(21) Appl. No.: 19/063,465

(22) Filed: Feb. 26, 2025

Related U.S. Application Data
(63) Continuation of application No. 18/049,544, filed on Oct. 25, 2022, now Pat. No. 12,245,873.
Application No. 18/049,544 is a continuation of application No. 15/692,681, filed on Aug. 31, 2017, now Pat. No. 11,490,858.
(60) Provisional application No. 62/382,043, filed on Aug. 31, 2016.

(51) Int. Cl. A61B 5/00; A61B 5/0205; A61B 5/11

(52) U.S. Cl. CPC A61B 5/6817; A61B 5/0002; A61B 5/1114; A61B 5/002; A61B 5/02055; A61B 2560/0214; A61B 2560/0425; A61B 2560/045

(57) ABSTRACT

A removable wearable device sleeve includes a sleeve body, one or more sensors, and an interface for operative communication with the wearable device. A removable sleeve includes a sleeve body having a front element having a first aperture, a back element having a second aperture, a tubular element having a first end and a second end with a third aperture, and one or more back elements define a cavity to encase an earpiece, an interface, and a sensor, wherein the sensor communicates sensor readings to the wearable device through the interface. A method of placing a sensor configured to communicate with a wearable device onto the wearable device includes providing a removable sleeve and inserting the wearable device through the first aperture of the front element into the cavity of the removable sleeve to provide a removable wearable device sleeve.

### PRIORITY STATEMENT

This application is a continuation of U.S. Non-Provisional patent application Ser. No. 18/049,544, filed on Oct. 25, 2022 which is a continuation of U.S. Non-Provisional patent application Ser. No. 15/692,681, filed on Aug. 31, 2017 now U.S. Pat. No. 11,490,858 which claims priority to U.S. Provisional Patent Application 62/382,043, filed on Aug. 31, 2016, all entitled Disposable Sensor Array Wearable Device Sleeve System and Method, hereby incorporated by reference in their entirety.

### FIELD OF THE INVENTION

The present invention relates to wearable devices. More particularly, but not exclusively, the present invention relates to earpieces.

### BACKGROUND

Sensors installed within electrical devices are prone to premature failure, requiring replacement of the nonfunctioning sensor or even the electrical device itself. Replacement of a defective sensor, however, may be difficult due to the fact that many sensors are either integrated with or contained within the electrical device. What is needed is an easily replaceable sensor or sensor array capable of operatively connecting to an electrical device while retaining its full functionality.

### SUMMARY

Therefore, it is a primary object, feature, or advantage of the present invention to improve over the state of the art.

It is a further object, feature, or advantage of the present invention to allow for cost-effective replacement of one or more sensors of a wearable device.

It is a still further object, feature, or advantage of the present invention to allow for replacement of one or more sensors of a wearable device without having to directly remove or replace a sensor installed within the wearable device.

Another object, feature, or advantage is to provide the ability to enhance one or more sensor functions of a wearable device.

Yet another object, feature, or advantage is to provide the ability to enhance one or more sensor functions of a wearable device without the need to replace a sensor directly installed on the wearable device.

In one implementation, a removable wearable device sleeve sized and shaped for fitting to a wearable device includes a sleeve body, one or more sensors integrated into the sleeve body, and an interface integrated into the sleeve body for operative communication between the at least one sensor and the wearable device.

One or more of the following features may be included. The wearable device may be an earpiece and the removable wearable device sleeve may be a removable earpiece sleeve. The removable earpiece sleeve may comprise a sleeve body having a front element having a first aperture, a back element having a second aperture, at least one side element, and a tubular element having a first end connected to the back element and a second end positioned away from the back element having a third aperture, wherein the front element, the back element, each side element, and the tubular element define a cavity to encase an earpiece. The removable earpiece sleeve may have one or more sensors integrated into the removable earpiece sleeve. One or more of the sensors may be a physiological sensor.

In another implementation, a removable sleeve includes a sleeve body having a front element having a first aperture, a back element having a second aperture, at least one side element, and a tubular element having a first end connected to the back element and a second end positioned away from the back element and having a third aperture, wherein the front element, the back element, each side element, and the tubular element define a cavity to encase an earpiece, an interface integrated into the sleeve body, and a sensor integrated into the removable sleeve, wherein the sensor communicates sensor readings to the wearable device through the interface. One or more of the following features may be included. The tubular element may further comprise a portion of the second aperture. The interface may provide for powering the sensor from the wearable device. The removable sleeve may be a removable earpiece sleeve.

In another implementation, a method of placing a sensor configured to communicate with a wearable device onto the wearable device includes providing a removable sleeve comprising the sensor, wherein the removable sleeve further comprises a front element having a first aperture, a back element having a second aperture, at least one side element, and a tubular element having a first end connected to the back element and a second end positioned away from the back element having a third aperture, wherein the front element, the back element, each side element, and the tubular element define a cavity; and inserting the wearable device through the first aperture of the front element into the cavity of the removable sleeve to provide a removable wearable device sleeve. One or more of the following features may be included. The wearable device may be an earpiece. The tubular element may further comprise a portion of the second aperture. The method may further comprise removing an old removable sleeve from the wearable device.

In another implementation, a removable sleeve includes a sleeve body having a front element having a first aperture, a back element having a second aperture, at least one side element, and a tubular element having a first end connected to the back element and a second end positioned away from the back element having a third aperture, wherein the front element, the back element, each side element, and the tubular element define a cavity to encase an earpiece and an interface integrated into the sleeve body. One or more of the following features may be included. The tubular element may further comprise a portion of the second aperture. The removable sleeve may be a removable earpiece sleeve.

One or more of these and/or other objects, features, or advantages of the present invention will become apparent from the specification and claims that follow. No single embodiment need provide each and every object, feature, or advantage. Different embodiments may have different objects, features, or advantages. Therefore, the present invention is not to be limited to or by an object, feature, or advantage stated herein.

### BRIEF DESCRIPTION OF THE DRAWINGS

FIG. 1 shows a block diagram of an embodiment of the removable wearable device sleeve.

FIG. 2 shows the sleeve interface and the types of interactions that may occur through the interface between a sensor integrated within the sleeve body and a wearable device.

FIG. 3 is an overhead view of a removable earpiece sleeve.

FIG. 4 is a front view of a removable earpiece sleeve.

FIG. 5 is a back view of a removable earpiece sleeve.

FIG. 6 is a top view of a removable earpiece sleeve.

FIG. 7 is a bottom view of a removable earpiece sleeve.

FIG. 8 is a left hand view of a removable earpiece sleeve.

FIG. 9 is a right hand view of a removable earpiece sleeve.

### DETAILED DESCRIPTION

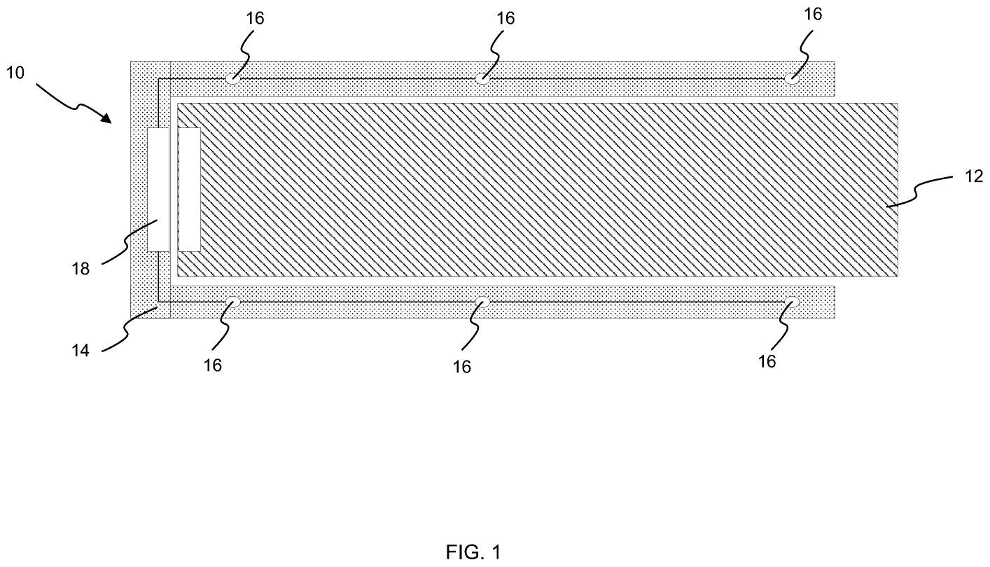

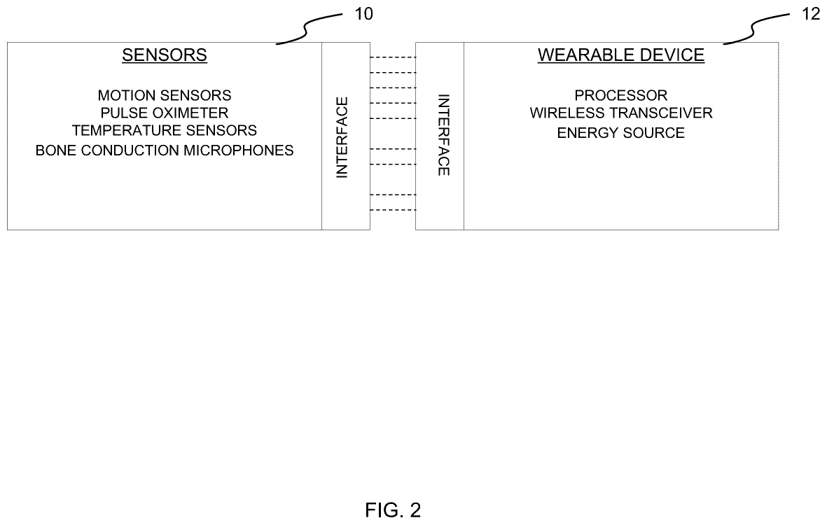

FIG. 1 shows a block diagram of one embodiment of the removable wearable device sleeve 10 and its relationship with a wearable device 12. The removable wearable device sleeve 10 comprises a sleeve body 14, at least one sensor 16 integrated into the sleeve body 14, and an interface 18 integrated into the sleeve body for operative communication between one or more sensors 16 and the wearable device 12. The wearable device 12 may fit substantially within the sleeve body 14 and may interact with the interface 18 of the removable wearable device sleeve 10 to provide energy or facilitate data transfer. The wearable device 12 may comprise one or more earpieces, a headset, headgear, belts, necklaces, bracelets, shirts, shoes, watches, legwear, or wearable rings, as seen in FIG. 2. The sleeve body 14 may be composed of one or more of any type of flexible material, which may include rubber, latex, synthetic plastic, natural fiber-based materials, plexiglass, or any other type of organic or synthetic polymer. More than one type of material may be used in order to take advantage of the different properties of each material. For example, an inner layer of the sleeve body 14 may be composed of latex while an outside layer may be composed of rubber in order to mitigate the chance a user may have an allergic reaction due to the presence of the latex. In addition, the sleeve body 14 may be composed of materials having a high tensile strength to resist tearing due to either repeated attachment and removal of the removable wearable device sleeve 10 or overstretching of the sleeve body 14 itself. The sensors 16 may be integrated into the sleeve body 14 at any position within the sleeve body 14 conducive to making a sensor reading. The sensors 16 integrated within the sleeve body may include a bone microphone, a temperature sensor, a pulse oximeter, a motion sensor, or any combination thereof, and the list is non-exclusive. One or more sensors 16 may also work together to enhance the functionality of a wearable device 12. For example, one of the sensors 16 may be an EMG sensor calibrated to sense electrical activity in the user's facial muscles, while a magnetometer may sense one or more head movements, wherein the sensor readings from both sensors may be communicated to the wearable device 12 to be used to calibrate a facial gesture used to control one or more functions of the wearable device 12. In addition, the sensors 16 may comprise part of a sensor installed on the wearable device 12 or may work in tandem with one or more sensors of the wearable device 12. For example, a removable wearable device sensor 16 may be a temperature sensor and a sensor installed on the wearable device 12 may be a humidity sensor, wherein each sensor work in tandem to provide localized weather information to the user of the wearable device. An interface 18 may be integrated into the sleeve body 14 and may provide for data communication and power. For example, one or more sensors 16 may communicate sensor readings across the interface to the wearable device 12 and the wearable device may provide electrical power to one or more of the sensors 16 integrated within the sleeve body 14.

FIG. 2 illustrates a block diagram of an interface 18 interacting with an interface of a wearable device 12. The interface 18 may communicate data from one or more sensors to the wearable device 12, and the data may be communicated continuously or discretely. The data communicated to the wearable device may comprise sensor readings related to temperature, humidity, user motion, motion related to outside objects, a user's heart rate, internal body sounds, luminescence, or any other number of indicia which may be of interest to the user or a third party. One or more different types of sensors readings may be communicated. For example, one sensor 16 (e.g. a bone conduction microphone) on the sleeve body 14 positioned proximate to a user's ear may sense internal body sounds for audio transparency purposes and communicate the sensor readings continuously, another sensor 16 (e.g. an MEMS gyroscope) may sense head and neck movement, which may be communicated discretely as the user moves his or her head, and another sensor 16 positioned on a portion of the sleeve body 14 to be inserted into a user's ear canal (e.g. a pulse oximeter) may communicate sensor readings related to a user's heartbeat. Data may also be communicated from the wearable device 12 to one or more sensors 16 via the interface 18 as well. For example, the wearable device 12 may communicate one or more position readings to a motion sensor (e.g. a MEMS gyroscope) 16 in order to properly calibrate the sensor 16 to sense user movements. Energy may also be transmitted via the interface 18 in order to power one or more sensors 16. For example, a battery, a capacitor, or an inductor installed within the wearable device 12 may supply power to one or more sensors 16 on either a continuous or as-needed basis depending on the setup of the wearable device 12 and/or the needs of the sensors 16. One or more sensors 16 may also obtain power via a voltage generated by thermal differences between the sleeve body 14, the sensors 16, or the interface 18 and the wearable device 12 or the user's body. It should be noted that the sensors may obtain power from sources other than the wearable device 12, such as through radio waves present in the user's surroundings or via solar apparatuses integrated within the sleeve body 14 which may convert sunlight into electricity that can be used to power one or more sensors 16.

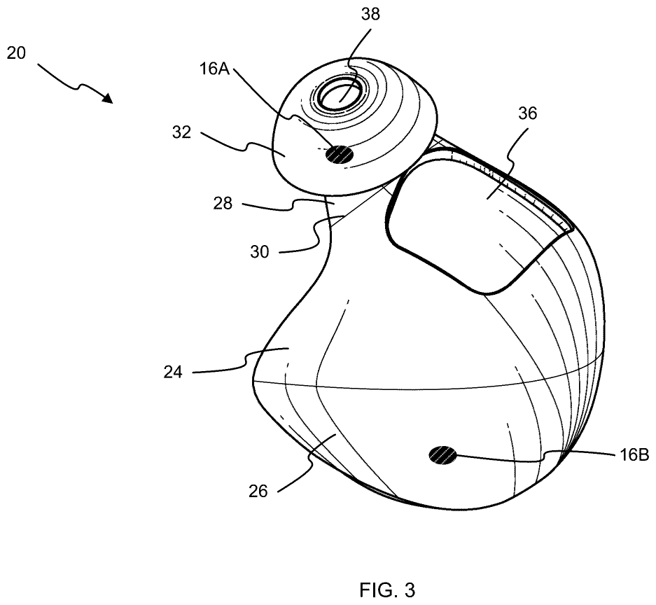

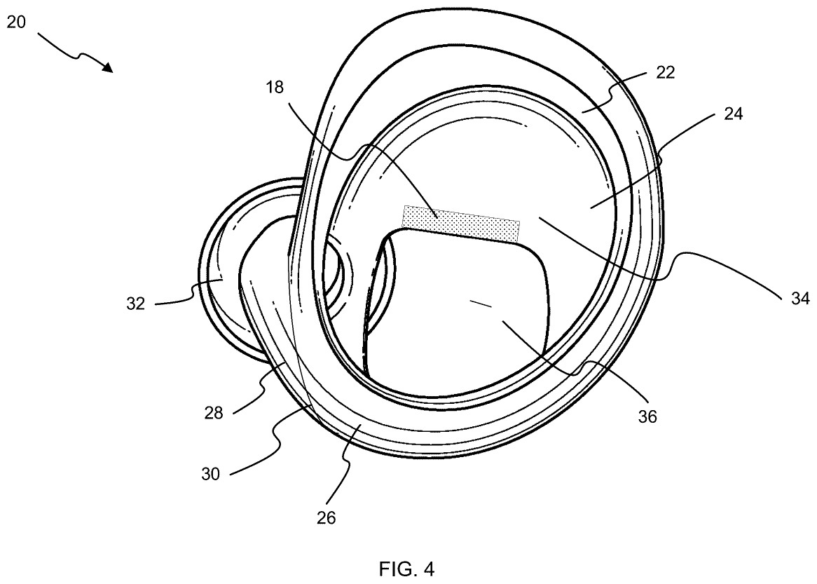

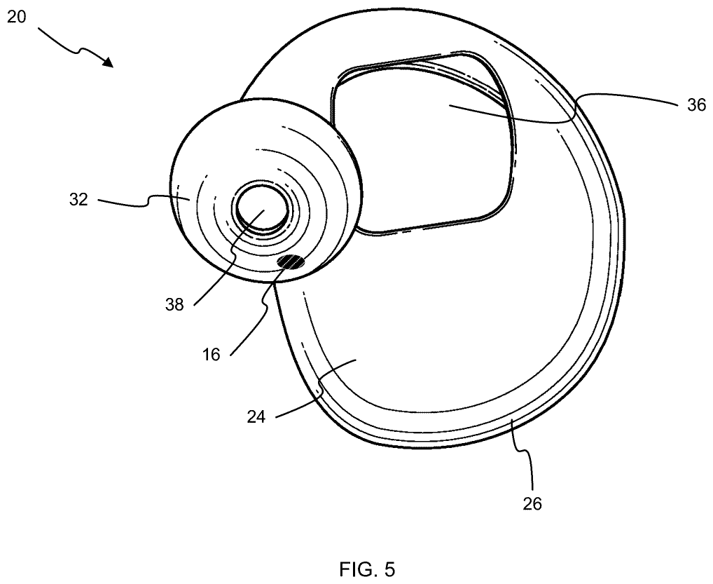

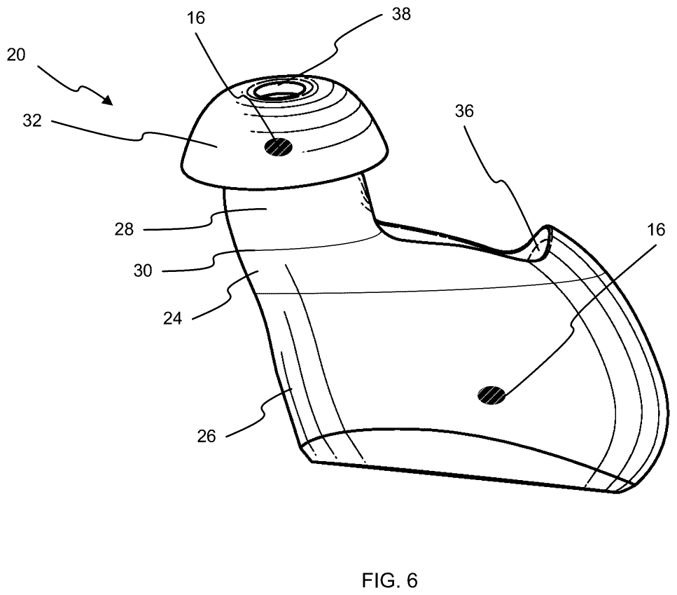

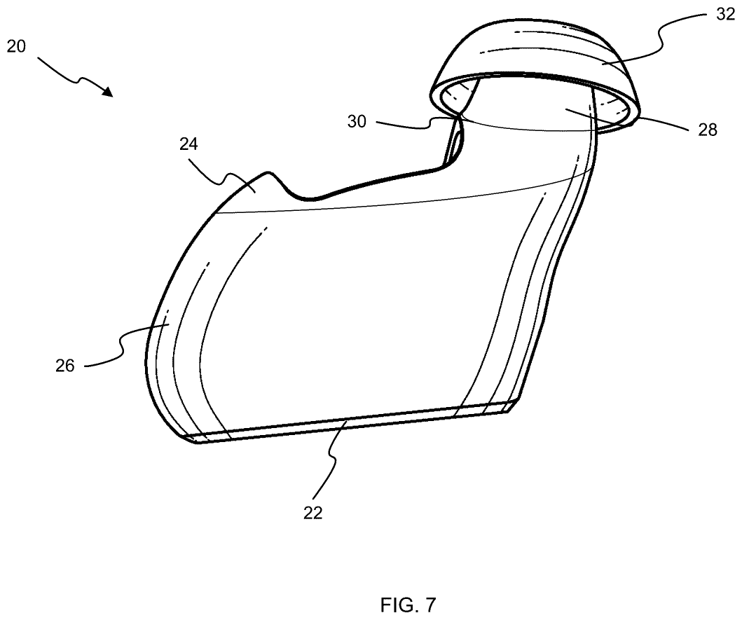

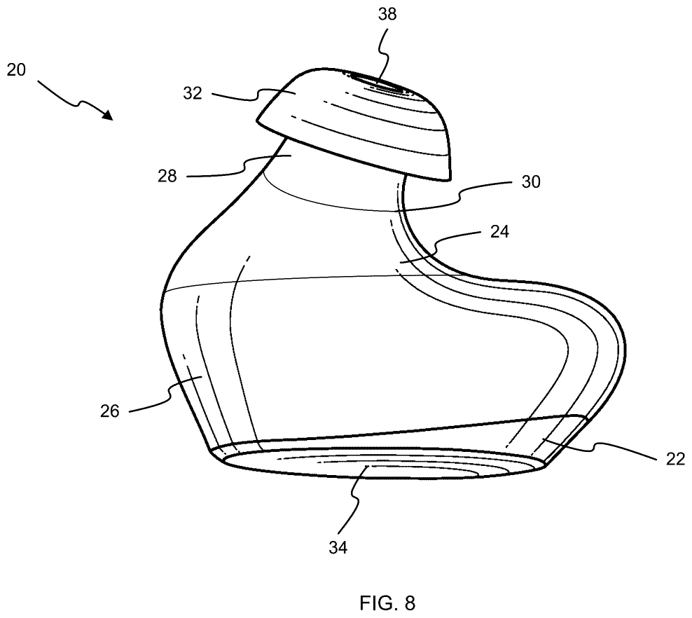

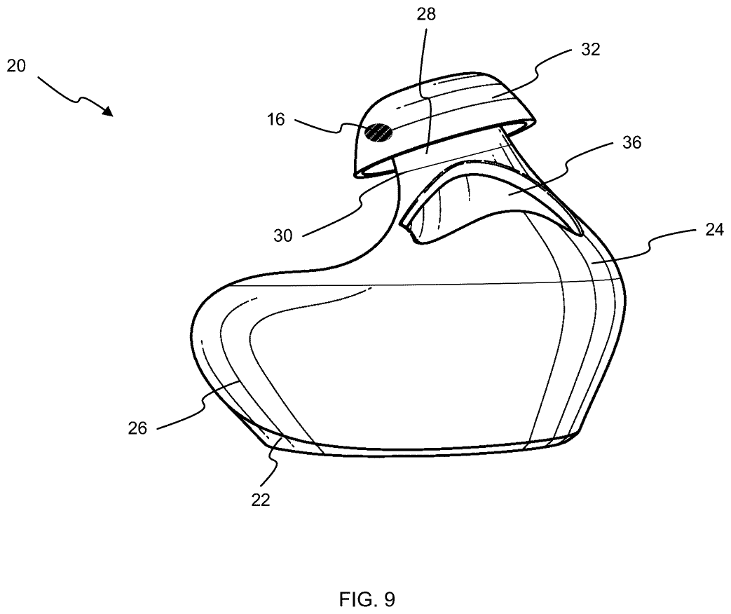

The following figures each show a removable earpiece sleeve 20 from various angles, one embodiment of a removable wearable device sleeve 10. FIG. 3 shows an overhead view of the removable earpiece sleeve 20, FIG. 4 shows a front view of the removable earpiece sleeve 20, FIG. 5 shows a back view of the removable earpiece sleeve 20, FIG. 6 shows a top view of the removable earpiece sleeve 20, FIG. 7 shows a bottom view of the removable earpiece sleeve 20, FIG. 8 shows a left side view of the removable earpiece sleeve 20, and FIG. 9 shows a right side view of the removable earpiece sleeve 20. The removable earpiece sleeve 20 comprises a front element 22 having a first aperture 34, a back element 24 having a second aperture 36, one or more side elements 26, and a tubular element 28 having a first end 30 connected to the back element 24 and a second end 32 positioned away from the back element 24 having a third aperture 38, wherein the front element 22, the back element 24, each side element 26, and the tubular element 28 define a cavity to encase an earpiece. The first aperture 34 is at a distal end of the sleeve, so for example, when the sleeve is placed on an earpiece which is then fitted to a user, the first aperture 34 is at a distal end of the device, facing away from the user. The third aperture 38 would similarly be at the proximal end of the sleeve, inserted into an external auditory canal of the user.

The removable earpiece sleeve 20 may also have one or more sensors 16 positioned throughout the sleeve 20. It may be noted, however, that the removable earpiece sleeve 10 does not need sensors installed within the sleeve. The perimeter of the front element 22 may be any size suitable to allow a user to see the front of an earpiece when an earpiece is encased within the removable earpiece sleeve 20. The first aperture 34 may be any size suitable for an earpiece to fit through and may substantially encompass the front element 22. The back element 24 may be sized to be smaller than or substantially similar to the front element 22, though a back element 24 sized substantially larger than the front element 22 would make it difficult to remove an earpiece from the removable earpiece sleeve 20 via the first aperture 34. The back element may have a second aperture 36 through which an earpiece may interface with a smart case or another electronic device capable of providing data communication, data transfer, and/or power to the earpiece. The second aperture 36 may also provide for user interaction with the earpiece, such as gesture control. In addition, the second aperture 36 may extend into the tubular element 28 as well. One or more side elements 26 connect the front element 22 to the back element 24, and may range from one side element (e.g. a cylindrical shaped sleeve) to theoretically hundreds of elements, and each side element 26 need not connect the front element 22 to the back element 24; the side elements 26 may, for example, be composed of a plurality of hexagons, where only the top layer and bottom layer of hexagons directly touches the front element 22 or the back element 24. The tubular element 28 is connected to the back element 24 via a first end 30 with a second end 32 positioned away from the back element 24 and having a third aperture 38. The tubular element 28 may be sized to fit a component of an earpiece designed to fit within a user's ear canal, and the third aperture 38 may provide an avenue for sound communicated by an earpiece to reach the user's tympanic membrane. The front element 22, the back element 24, each side element 26, and the tubular element 28 may define a cavity for which to encase an earpiece. The cavity may be shaped to fit a specific earpiece or may be shaped to partially or substantially encase earpieces of different sizes or shapes, and the earpiece may be placed into the cavity through the first aperture 34. If the user wishes to replace the removable earpiece sleeve 20 for any reason, for example, if one of the sensors on either the earpiece or the removable earpiece sleeve 20 suffers a loss of functionality or if the sleeve suffers a loss of functionality due to wear and tear, the earpiece may be removed through the first aperture 34, through the second aperture 36, or though ripping or tearing the removable earpiece sleeve 20 off of the earpiece. In addition, a removable earpiece sleeve 20 may be replaced if the user desires to improve or alter the functionality of an earpiece through the use of one or more new sensors on a new removable earpiece sleeve 20 even if the sensors in the original sleeve and the earpiece are still functional.

It should also be understood that various sensors may be positioned on different portions of the sleeve. For example, in FIG. 3 a sensor 16A such as a physiological sensor is positioned near the second end 38 and may be positioned to fit against a wall of an external auditory canal of a user. Another sensor 16B which may also be a physiological sensor may be placed more distally.

A method placing a sensor configured to communicate with a wearable device onto the wearable device is provided. First, a removable sleeve having at least one or more sensors is provided, wherein the removable sleeve further comprises a front element having a first aperture, a back element having a second aperture, at least one side element, and a tubular element having a first end connected to the back element and a second end positioned away from the back element and having a third aperture, wherein the front element, the back element, each side element, and the tubular element define a cavity. The removable sleeve may be composed of one or more of any type of flexible material that has sufficient tensile strength to resist tearing and is resistant to fatigue, and each sensor may be positioned at any location on the removable sleeve conducive to facilitating a sensor reading. The first aperture may be sized to allow a wearable device to be inserted into the cavity, and the second aperture may permit the wearable device to interface with one or more electronic devices to, for example, recharge or transfer data. The front element, the back element, and each side element may be sized to fit a specific wearable device or to partially or substantially encase a wearable device, and more than one side element may be present. The tubular element may be located at any position on the back element and the third aperture may help facilitate audio communication from a speaker or communication unit of a wearable device if a wearable device is fitted to the removable sleeve. If a sleeve is already present on the wearable device, the old sleeve is removed from the wearable device. The old sleeve may be removed due to the malfunction or failure of one or more sensors on either the old sleeve or the wearable device, the malfunction or failure of one or more functions of the wearable device, or a mechanical failure of the old sleeve itself. If a sleeve was not already present on the wearable device, or if a sleeve was present on the wearable device and removed, then, the wearable device may be inserted into the cavity of the removable sleeve through the first aperture to provide a removable wearable device sleeve. The cavity may be shaped to fit the wearable device or may be shaped to approximate a fit for a random wearable device, and the cavity may encase only a portion of the wearable device. In addition, the removable wearable device sleeve may enhance one or more sensor functions of the wearable device when worn, and may also streamline one or more functions of the wearable device. Therefore, a removable wearable device sleeve and related methods have been shown and described.

What is claimed is:

1. A system comprising:
a wireless earpiece configured to fit within an ear of a user, the wireless earpiece comprising a wireless earpiece body, a first interface, and a motion sensor integrated into the wireless earpiece body;
a removable sleeve comprising a sleeve body formed of a flexible material, the sleeve body defining a cavity configured to encase the wireless earpiece body such that the removable sleeve contacts the ear of the user when the wireless earpiece is worn;
a second interface integrated into the sleeve body and configured to operatively connect with the first interface of the wireless earpiece; and
a plurality of physiological sensors integrated into the sleeve body, wherein the plurality of physiological sensors are electrically connected to the second interface and configured to communicate physiological sensor readings to the wireless earpiece via the first and second interfaces, and wherein the wireless earpiece is configured to process the physiological sensor readings in combination with motion data from the motion sensor when the removable sleeve is fitted to the wireless earpiece and worn by the user.

2. The system of claim 1, wherein the plurality of physiological sensors includes a bone microphone positioned on the sleeve body such that, when the removable sleeve is fitted to the wireless earpiece and worn by the user, the bone microphone is proximate to an external auditory canal of the user to detect internal body sounds.

3. The system of claim 2, wherein the wireless earpiece is configured to process the internal body sounds detected by the bone microphone to provide audio transparency for the user.

4. The system of claim 1, wherein the plurality of physiological sensors includes a pulse oximeter positioned on the sleeve body such that, when the removable sleeve is fitted to the wireless earpiece and worn by the user, the pulse oximeter contacts a wall of the external auditory canal of the user.

5. The system of claim 1, wherein the plurality of physiological sensors includes a temperature sensor configured to measure a body temperature of the user when the removable sleeve is fitted to the wireless earpiece and worn by the user.

6. The system of claim 1, wherein the plurality of physiological sensors includes a pulse oximeter, and the wireless earpiece is configured to integrate pulse oximetry data from the pulse oximeter with motion data from the motion sensor to determine an activity-adjusted physiological metric for the user.

7. The system of claim 1, wherein the second interface is configured to receive power from the wireless earpiece via the first interface to power the plurality of physiological sensors.

8. The system of claim 1, wherein the sleeve body comprises a front element having a first aperture, a back element having a second aperture, at least one side element, and a tubular element having a first end connected to the back element and a second end positioned away from the back element having a third aperture, wherein the cavity is defined by the front element, the back element, the at least one side element, and the tubular element.

9. The system of claim 8, wherein at least one of the plurality of physiological sensors is positioned along the tubular element to contact an external auditory canal of the user when the wireless earpiece is worn.

10. The system of claim 1, wherein the flexible material of the sleeve body comprises a rubber layer and a latex layer, the latex layer positioned as an inner layer facing the cavity and the rubber layer positioned as an outer layer.

11. A method of customizing physiological monitoring for a wireless earpiece, the method comprising:
providing a wireless earpiece comprising a wireless earpiece body, a first interface, and a motion sensor integrated into the wireless earpiece body;
selecting a first removable sleeve from a plurality of removable sleeves based on a desired physiological monitoring function, the first removable sleeve comprising a first sleeve body formed of a flexible material, a second interface integrated into the first sleeve body, and a first set of physiological sensors integrated into the first sleeve body, wherein the first sleeve body defines a cavity;
inserting the wireless earpiece into the cavity of the first removable sleeve through an aperture in the first sleeve body, such that the first set of physiological sensors communicates first physiological sensor readings to the wireless earpiece via the first and second interfaces when the wireless earpiece is worn by a user; removing the first removable sleeve from the wireless earpiece;
and inserting the wireless earpiece into a cavity of a second removable sleeve comprising a second sleeve body formed of a flexible material, a third interface integrated into the second sleeve body, and a second set of physiological sensors integrated into the second sleeve body, wherein at least one physiological sensor in the second set differs in type from the first set, and the second set of physiological sensors communicates second physiological sensor readings to the wireless earpiece via the first and third interfaces when the wireless earpiece is worn by the user.

12. The method of claim 11, wherein the first set of physiological sensors includes a temperature sensor and the second set of physiological sensors includes a pulse oximeter.

13. The method of claim 11, wherein removing the first removable sleeve is performed in response to a malfunction of at least one sensor in the first set of physiological sensors.

14. The method of claim 11, further comprising processing, by the wireless earpiece, the first physiological sensor readings in combination with motion data from the motion sensor to generate a first physiological metric, and processing the second physiological sensor readings in combination with motion data to generate a second physiological metric different from the first physiological metric.

15. The method of claim 11, wherein removing the first removable sleeve comprises extracting the wireless earpiece through the aperture in the first sleeve body.

16. A system for physiological monitoring with a wireless earpiece, comprising:
a wireless earpiece comprising a wireless earpiece body, a first interface, and a motion sensor integrated into the wireless earpiece body;
a first removable sleeve comprising a first sleeve body formed of a flexible material defining a cavity configured to encase the wireless earpiece body, a second interface integrated into the first sleeve body, and a first set of physiological sensors integrated into the first sleeve body including at least a temperature sensor;
a second removable sleeve comprising a second sleeve body formed of a flexible material defining a cavity configured to encase the wireless earpiece body, a third interface integrated into the second sleeve body, and a second set of physiological sensors integrated into the second sleeve body including at least a pulse oximeter and excluding a temperature sensor;
wherein each of the first and second removable sleeves is configured to operatively connect to the wireless earpiece via the first interface, such that the respective sets of physiological sensors communicate physiological sensor readings to the wireless earpiece for processing with motion data from the motion sensor when the wireless earpiece is worn by a user.

17. The system of claim 16, wherein the second set of physiological sensors further includes a bone microphone.

18. The system of claim 16, wherein each of the first sleeve body and the second sleeve body comprises a front element having a first aperture, a back element having a second aperture, at least one side element, and a tubular element defining the respective cavity.

19. The system of claim 16, wherein the temperature sensor of the first removable sleeve and the pulse oximeter of the second removable sleeve are positioned to contact an external auditory canal of the user when the respective removable sleeve is fitted to the wireless earpiece and worn by the user.

20. The system of claim 16, wherein the wireless earpiece is configured to process temperature sensor readings from the first removable sleeve with motion data to output a body temperature metric, and to process pulse oximetry readings from the second removable sleeve with motion data to output an oxygen saturation metric.
